(19) United States
(12) Patent Application Publication
(10) Pub. No.: US 2005/0170064 A1
(43) Pub. Date: Aug. 4, 2005

(54) Plant sterol-containing food, and method for preparing the same

(75) Inventors: Won-Tae Yoon, Seoul (KR); Kab-Sig Kim, Koyang (KR); Bo-Chun Kim, Kimpo (KR); Jung-Hee Han, Seoul (KR)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Eugene Science Inc., Seoul (KR)

(21) Appl. No.: 10/508,689

(22) PCT Filed: Mar. 20, 2002
(86) PCT No.: PCT/KR02/00472

(51) Int. Cl. A23D 7/06; A61K 31/56

(52) U.S. Cl. 426/604; 514/169

(57) ABSTRACT

Disclosed are the plant sterol-containing food, and methods for preparing the same. The particle size of the plant sterol contained in the food is nanometer-scale, and the plant sterol-containing food of the present invention inhibits the absorption of cholesterol.

### TECHNICAL FIELD

The present invention relates to plant sterol-containing foods and a method for preparing the same. More particularly, the present invention relates to foods containing hundreds of nanometers of plant sterol micelles or less to inhibit the absorption of cholesterols into the body, and a method for preparing the same.

### BACKGROUND ART

Found in large concentrations in the brain, nervous tissues, organs, and blood plasma of higher animals, cholesterol, a kind of steroids, is the major precursor of the synthesis of vitamin D and various steroid hormones, including sex hormones (testosterone, progesterone, etc.,), adrenal cortical hormone, bile acid, etc. High levels of cholesterol in the blood are associated with an increased risk of cardiovascular diseases, such as hyperlipidemia, arteriosclerosis, arrhythmia, cardiac infarction, and so on. As a result of over-ingestion of cholesterol, diseases associated with cholesterol are becoming an increasingly big social problem.

It is known that both endogenic and dietary cholesterol move into the small intestine and about 50% thereof is absorbed from the intestines (Bosner, M. S., Ostlund, R. E., Jr., Osofisan, O., Grosklos, J., Fritschle, C., Lange, L. G. 1993). Based on this fact, a mechanism for preventing cholesterol from being absorbed from intestines is of special interest to those who have made efforts to discover clues for the prophylaxis and treatment of cholesterol-associated diseases.

Naturally occurring in a broad spectrum of plants such as bean, corn, wood, tallow oil, etc., plant sterol (or phytosterol) or plant stanol (or phytostanol) is non-toxic. The plant sterol or phytosterol includes sitosterol, campesterol and stigmasterol, while the plant stanol or phytostanol includes sitostanol and campestanol. For purposes of convenience, they are all called plant sterol herein.

With structures very similar to that of cholesterol, plant sterol, when ingested in large quantities, is known to inhibit the absorption of intestinal and bile cholesterol, thereby reducing the serum cholesterol level, as disclosed in U.S. Pat. No. 5,578,334. By taking advantage of the inhibitory function of plant sterol against cholesterol absorption, clinical trials have been conducted of plant sterol as a therapeutic agent for treatment of cardiovascular diseases, coronary artery diseases and hyperlipidemia (Atherosclerosis 28:325-338).

Despite this useful function, the plant sterol is difficult to apply to foods on account of its physical properties, that is, very poor solubility in both water and oil. Accordingly, there have been developed foods with only limited content of plant sterol.

With the aim of increasing the solubility of plant sterol, some researchers have synthesized various derivatives of the plant sterol. For example, ester forms of the plant sterol were developed, which have excellent solubility in oil phases (Mattson F. H., R. A. Volpenhein, and B. A. Erickson, 1997). In U.S. Pat. No. 5,502,045, sitostanol fatty acid ester is disclosed which is prepared by the interesterification of sitostanol with a fatty acid ester. According to this patent, the sitostanol fatty acid ester is reported to reduce the LDL-C level by as much as 16% when being used in an applied form in an oil phase (margarine).

PCT WO 99/15546 and WO 99/15547 describe water- and oil-soluble plant sterol derivatives which are synthesized by linking a water- or oil-soluble molecule to the plant sterol or the plant stanol via an ester linkage.

However, one research result reveals that synthetic plant sterol derivatives with improved solubility have lower inhibitory effects on intestinal cholesterol absorption than does natural plant sterol (Mattson et al., The American Journal of Clinical Nutrition 35: April 1982 pp 697-700).

In addition to the effort to increase the solubility of plant sterol through the synthesis of derivatives, intensive research has been and continues to be directed to improving the bioavailability of plant sterol.

For example, U.S. Pat. No. 5,932,562 discloses an aqueous homogeneous micellar mix of a plant sterol, lecithin and lysolecithin, which has been dried to a finely divided water soluble powder. This was obtained by mixing plant sterol, lecithin and lysolecithin together in chloroform at a fixed molar ratio and removing the chloroform therefrom. In this patent, however, some problems are inherent. The total amount of the emulsifiers used in the patent is greater than that of the plant sterol. The emulsifier lysolecitin is very expensive. What is worse, the organic solvent used to form the micelles makes the water-soluble powder unsuitable for ingestion.

Other water-soluble plant sterols can be found in U.S. Pat. Nos. 6,054,144 and 6,110,502. According to these patents, aqueous-dispersible plant sterol is produced by admixing oryzanol or plant sterol, a mono functional surfactant and polyfunctional surfactant in water at fixed ratios, and drying the admixture. This production method is characterized by being free from homogenization and deaeration steps with adoption of polyoxylene sorbitan monopalmitate and sorbitan monopalmitate as a mono functional surfactant and a polyfunctional surfactant, respectively.

In European Pat. Publication No. 289,636 is described a method of producing emulsified or solubilized sterol in a stable form by admixing plant sterol at a fixed ratio with a liquid polyhydroxy compound containing sucrose fatty acid ester and/or polyglycerol fatty acid ester and diluting the admixture.

U.S. Pat. No. 6,190,720 discloses a food ingredient that can be used as a cholesterol-lowering agent, teaching that the food ingredient can be prepared by combining one or more molten plant sterols with one or more fats and one or more emulsifiers to homogeneity and cooling the homogeneous mixture to about 60° C. under agitation to give a paste. This food ingredient can be applied to oil-based foods such as salad dressings, margarine, etc.

EP 0 897 671 A1 is directed to aqueous dispersions of plant sterols useful in spreads, dressings, milk, cheese, etc, and to a preparation method comprising mixing together a molten high melting point lipid, a non-sterol emulsifier and water under shear, with a condition that the high melting point lipid has a mean size of 15 microns or less. This technique enjoys the advantage of allowing for minimization or elimination of use of saturated fatty acids and unsaturated fatty acids during the preparation.

Cholesterol reducing, edible products can be found in PCT WO 00/33669. According to the method of this prior art, plant sterols are dissolved or mixed in a melt of a food emulsifier, admixed with protein-containing foods such as milk or yogurt, homogenized, and added to food products. The dispersion stability of the cholesterol reducing, edible products is maintained only in the presence of a protein-containing material, but not maintained in the absence of a protein-containing material. Therefore, it is very difficult to apply the cholesterol reducing, edible products to foods free of protein.

U.S. Pat. No. 6,267,963 is concerned with a plant sterol-emulsifier complex which has a melting temperature at least 30° C. below that of the plant sterol, characterized in that, due to its reduced melting temperature, the plant sterol-emulsifier is less likely to crystallize during or after the manufacture of food products, and can be incorporated into food products in an amount effective to reduce serum cholesterol levels in a human consuming the food products without unpleasant effects on the texture of the food products.

However, plant sterol-containing foods prepared according to the aforementioned conventional techniques show disadvantages that micelle particles of the plant sterol produced amount, in size, to as large as tens of micrometers, being bristly to the feel of the mouth, and are poor in terms of long-term stability due to phase separation or water separation.

### DISCLOSURE OF THE INVENTION

With the problems in mind, the intensive and thorough research on soluble forms of plant sterols, conducted by the present inventors, resulted in the finding that the size of the dispersed particles of plant sterol determines the dispersion stability and bio-availability of plant sterol and that reduction of the size to nanometer levels is a solution for the above problems encountered in prior arts. It was also found that, in foods, dispersed particles with a size of hundreds nanometers or less of plant sterol are superior in bio-availability, having no influence on the characteristic taste and flavor of the foods, in addition to being applied to almost all foods irrespective of food bases and pH, and the improvement in the dispersion stability of the nano-sized plant sterol particles has the effect of prolonging the life span of the food, guaranteeing the stability of the products for a long period of time. A clue came from the finding that, when plant sterol and at least one emulsifier selected from the group consisting of sucrose fatty acid ester, sorbitan fatty acid ester and polyglycerin fatty acid ester, are heated together in the absence of other additional components, they are brought into homogeneous contact with each other while being fused, to form fine micelles which are as small as nanometers in size, at subsequent high-speed stirring or homogenizing processes, leading to the present invention.

Therefore, the object of the present invention is to provide a method for preparing plant sterol-containing foods, in which the plant sterol is dispersed in such a nano-sized level that it is improved in bio-availability and can be applied to various foods, irrespective of food bases and pH, with no influence on the characteristic taste and flavor of the applied food, and without providing a bristly sensation in the mouth.

It is another object of the present invention to provide a plant sterol-containing food, which can inhibit the absorption of intestinal cholesterol and bile cholesterol even when it is ingested in a relatively small amount thanks to the high bio-availability of the plant sterol contained therein, in addition to not producing a bristly sensation in the mouth.

In accordance with a first embodiment of the present invention, there is provided a method for preparing a plant sterol-containing food, comprising the steps of:

thermally melting an admixture of plant sterol and at least one emulsifier at 60-200° C., said emulsifier being selected from the group consisting of sucrose fatty acid esters, sorbitan fatty acid esters and polyglycerine fatty acid esters;

combining the molten admixture with water or emulsifier-containing water;

stirring the combination at a high speed to give a dispersion of plant sterol of micelle form in water; and

applying the dispersion to a food base, the plant sterol being dispersed into particles with a size of hundreds of nanometers or less in the food base.

In accordance with a second embodiment of the present invention, there is provided a method for preparing a plant sterol-containing food, comprising the steps of:

thermally melting an admixture of plant sterol and an emulsifier at 60-200° C., said emulsifier being selected from the group consisting of sucrose fatty acid esters, sorbitan fatty acid esters and polyglycerine fatty acid esters;

combining the molten admixture with water or emulsifier-containing water;

stirring the combination at a high speed, followed by homogenizing to give a dispersion of plant sterol of micelle form in water; and

applying the dispersion to a food base, the plant sterol being dispersed into particles with a size of hundreds of nanometers or less in the food base.

In accordance with a third embodiment of the present invention, there is provided a method for preparing a plant sterol-containing food, comprising the steps of:

thermally melting an admixture of plant sterol and at least one emulsifier at 60-200° C., said emulsifier being selected from the group consisting of sucrose fatty acid esters, sorbitan fatty acid esters and polyglycerine fatty acid esters;

cooling the molten admixture for solidification; pulverizing the solidified admixture into powders, and combining the powders with water or emulsifier-containing water;

stirring the combination at a high speed to give a dispersion of plant sterol of micelle form in water; and

applying the dispersion to a food base, the plant sterol being dispersed into particles with a size of hundreds of nanometers or less in the food base.

In accordance with a fourth embodiment of the present invention, there is provided a method for preparing a plant sterol-containing food, comprising the steps of:

thermally melting an admixture of plant sterol and at least one emulsifier at 60-200° C., said emulsifier being selected from the group consisting of sucrose fatty acid esters, sorbitan fatty acid esters and polyglycerine fatty acid esters;

cooling the molten admixture for solidification, pulverizing the solidified admixture into powders, and combining the powders with water or emulsifier-containing water;

stirring the combination at a high speed, followed by homogenizing to give a dispersion of plant sterol of micelle form in water; and

applying the dispersion to a food base, the plant sterol being dispersed into particles with a size of hundreds of nanometers or less in the food base.

In accordance with a fifth embodiment, there is provided a plant sterol-dispersed food prepared by one of the above methods.

### BEST MODES FOR CARRYING OUT THE INVENTION

Plant sterols are naturally occurring materials similar in structure to cholesterol. In the natural world, there are found a variety of plant sterols, of which sitosterol, campesterol, stigmasterol and sitostanol predominate over other sterols. In the present invention, the term “plant sterol” refers to all sterols and stanols found in plants, including sitosterol, campesterol, stigmasterol, sitostnaol, campestanol, etc.

Many attempts have been made to apply plant sterols to foods. International patent application No. PCT/KR01/01640, filed by the present inventors, discloses a method of dispersing plant sterols into micelle with a size of nanometers level, whose content is incorporated herein by reference.

According to the present invention, plant sterol is admixed in a suitable ratio with at least one specific emulsifier and then the admixture is heated and melted as the first step for the preparation of the plant sterol-containing foods.

In this regard, useful are emulsifiers capable of dispersing plant sterols in a form of micelles with a size of hundreds of nanometers or less and whose examples include sucrose fatty acid ester, sorbitan fatty acid ester, and polyglycerine fatty acid ester. Other emulsifiers than sucrose fatty acid ester, sorbitan fatty acid ester, and polyglycerine fatty acid ester were found to give significant amounts of particles with a size of 1 micrometer or larger, as measured by various experiments. In practice, the other emulsifiers showed such low dispersion stability as to cause precipitates or water separation within three days after the dispersion of plant sterols therewith. Accordingly, the emulsifiers except for sucrose fatty acid ester, sorbitan fatty acid ester and polyglycerine fatty acid ester are not desirable to apply for the preparation of foods. Preferable among sucrose fatty acid esters are those that have a hydrophilic lypophilic balance (HLB) value of 7 or higher. Their HLB values are more preferably 10 to 16. Sorbitan fatty acid esters preferably have an HLB value of 5 to 11 and more preferably 7 to 10. As for polyglycerine fatty acid esters, their HLB values preferably range from 10 to 20 and more preferably from 12 to 15. Sucrose fatty acid esters produce smaller particles and more homogeneous particle size distribution than do the other emulsifiers, i.e., sorbitan fatty acid esters and polyglycerine fatty acid esters. In addition, sorbitan fatty acid esters and polyglycerine fatty acid esters emit a slight offensive odor when they are used in large quantities. Consequently, sucrose fatty acid esters are most preferable.

In the present invention, the weight ratio of plant sterols and total emulsifiers (including an emulsifier introduced by the combination with emulsifier-containing water) is in the range of 1:0.01 to 1:20 (w/w) and preferably in the range of 1:0.2 to 1:2.0 (w/w). As such, if the weight ratio of the emulsifiers to plant sterols is below 0.01, sufficient emulsification is not achieved, thus occurring precipitation, and the emulsified particles, if formed, amount to as large as tens of micrometers in size. On the other hand, if the weight ratio exceeds 20, the resulting food acquires the taste of the emulsifiers, being poor to the feel of the mouth.

In the case of employing the emulsifier-containing water, the emulsifier contained therein is used at a weight ratio of 0.8 or less of the emulsifier admixed with the plant sterols (i.e., 80% by weight or less based on the weight of the emulsifier admixed with the plant sterols) and preferably at a weight ratio of 0.5 or less (i.e., 50% by weight or less). A weight ratio of greater than 0.8 (w/w) (80% by weight) makes it difficult to form nano-sized particles because the amount of the emulsifier admixed with plant sterol is relatively low.

In accordance with the present invention, plant sterol and an emulsifier are admixed at 60-200° C. Preferable heating temperatures of the admixture fall within the range of 120-150° C. When the admixing is conducted at less than 60° C., the micelle particles have a size of from tens to hundreds of micrometers, thus being poor to the feel of the mouth as well as in bio-availability. On the other hand, an admixing temperature higher than 200° C. denatures the emulsifier even though the plant sterol is stable at 250° C.

Generally, when plant sterol, a sparingly water-soluble substance, is emulsified in water in the presence of an emulsifier, poor emulsification occurs, resulting in settling the plant sterol into particles with a size ranging from tens to hundreds of micrometers. While, in the present invention, the emulsification of plant sterol is maximized, thereby allowing the micelle to be in the particle size of hundreds of nanometers or less. For this, the emulsification should be conducted with the plant sterol and the emulsifier being mixed to homogeneity. In order to homogeneously mix the plant sterol with the emulsifier, the plant sterol is heated to near its melting point (sitosterol: about 140° C.; campesterol: about 157° C., stigmasterol: about 170° C.) to bring the two components into liquid phases before mixing.

In accordance with the present invention, the melted admixture is combined with water alone or emulsifier-containing water. This emulsifier is preferably the same as that admixed with the plant sterol. However, a different emulsifier may be used if they are compatible with each other. The weight ratio of the plant sterol and water falls within the range of 1:10 to 1:10,000 (w/w) and preferably within the range of 1:10 to 1:100 (w/w).

The combination composed of the melted mixture and water or emulsifier-containing water is stirred at high speeds and optionally homogenized to produce a dispersion in which nano-sized particles are formed. Resulting in homogeneous particle size distribution, the high-speed stirring (or the high-speed stiring and homogenizing) is industrially important in terms of consistent quality of products.

In this regard, the stirring is conducted at 5,000-10,000 rpm and preferably at 6,500-7,500 rpm for about 10 min. 90% or more, preferably 95% or more of the micelles thus obtained are in the size of 300 nm or less.

The homogenizing is optionally needed to pulverize some aggregated micelles, if any, after the stirring. This homogenizing step may be conducted with the aid of a high-pressure homogenizer, a colloid mill or a sonicator with preference for a high-pressure homogenizer. At that time, the micelles are homogenized at a pressure of 2,000-25,000 psi and preferably at a pressure of 7,000-10,000 psi in a homogenizer. 95% or more, preferably 99% or more of the micelles thus obtained are in the size of 300 nm or less.

Alternative embodiments of the present invention are described, below.

The plant sterol may be admixed with an emulsifier and heated at near the melting point thereof, and the melt admixture is cooled for the solidification, and then pulverized into powders. By stirring the powders at a high speed in water or emulsifier-containing water, an aqueous dispersion of plant sterol can be prepared. In this case, the homogenizing step may optionally follow the stirring step for removal of the aggregated micelles, as discussed earlier.

According to the present invention, when undergoing the high-speed stirring (in particular, when undergoing the high-speed stirring and the homogenizing steps), a clear plant sterol dispersion is formed. For comparison, when the plant sterol is used at an amount of 1%, the conventional emulsification cannot guarantee the dispersion stability of the resulting solution, giving rise to an increase in settling of plant sterol. While the conventional emulsification processes produce dispersions which show a transmittance at 700 nm of as low as 0.16%, the method according to the present invention promises a transmittance at 700 nm of 80.0% or higher.

In particular, the admixture of plant sterol and emulsifier in the powder form as mentioned above has advantages over liquid form in that it is convenient to handle, safer from microorganism contamination during transportation, and easy to transport with low logistics cost.

In accordance with the present invention, when combining the admixture of plant sterol and emulsifier with the water or emulsifier-containing water, the admixture of plant sterol and emulsifier may be in the form of a hot liquid phase or a solid phase cooled. At this time, the water or emulsifier-containing water is heated to preferably about 60-140° C. Even though the heating temperature of the water or emulsifier-containing water is preferably adjusted to near the admixing temperature of the plant sterol and the emulsifier to result in small micelle particles and enhancement of the emulsification efficiency, practically the temperature range, which the water or emulsifier-containing water is heated to, can be in the range of about 70-90° C. for the commercial convenience in production. In the case of raising the temperature of the water or emulsifier-containing water to higher than 100° C., the pressurization is required. For example, about 5 atm is required to heat the water or emulsifier-containing water to 140° C.

In contrast, the micelles obtained under the same conditions, except that the admixture of plant sterol and emulsifier is not heated, were measured to range, in particle size, from tens to hundreds of micrometers. Therefore, these comparative measurements demonstrate that the step of melt-mixing plant sterol and an emulsifier plays a very important role in forming nano-sized particles. Additionally, the high-speed stirring (or the high-speed stirring and homogenizing) is important in making particles homogeneous in size, as will be described later.

Upon heating in the absence of other components, the plant sterol and an emulsifier can be brought into homogeneous contact with each other while being melted, so that the micelles are obtained with a size of hundreds of nanometers after the emulsification. Contrary to the conventional techniques, the present invention is, therefore, capable of producing nano-sized particles suitable for use in foods without using any organic solvents in which plant sterol is fairly soluble.

The dispersion obtained after the admixture of plant sterol and emulsifier is dispersed in water, is evaporated and freeze-dried or spray-dried to produce a water-soluble plant sterol powders. These powders may be dispersed again in water and applied to foods.

The dispersion obtained in accordance with the present invention, is applied to food bases to afford desired plant sterol-containing foods. In these foods, the micelles with a particle size as small as hundreds of nanometers or less have large surface areas and particle curvatures, and thus are superior in bio-availability, having no influence on the characteristic taste and flavor of the foods. Additionally, the foods according to the present invention do not undergo layer separation, nor water separation even after being stored in a refrigerator because the plant sterol micelles are improved in dispersion stability. Further, the plant sterol micelles maintain excellent dispersion stability in foods which are stored at a warm temperature upon marketing, guaranteeing the stability of the products for a long period of time.

Examples of non-beverage food bases to which the plant sterol dispersion is applicable, include yogurt, gruel, soup, ice cream, mayonnaise, ketchup, cheese, salad oil, dressings, and margarine.

Having generally described this invention, a further understanding can be obtained by reference to certain specific examples which are provided herein for purposes of illustration only and are not intended to be limiting unless otherwise specified. In the following examples, particle size distribution was analyzed by use of Mastersizer (Malvern Instrument LTD., UK).

### COMPARATIVE EXAMPLE 1

To 1 liter vessel was added 500 g of water which was then heated to about 80° C. 5 g of plant sterol (sitosterol 75%, campesterol 10%, and stigmasterol and sitostanol 15%) and 4.25 g of sucrose stearyl ester (HLB 11) were added to the heated water, followed by stirring the mixture at a speed of 6,800-7,000 rpm for 10 min. The particles thus obtained were analyzed for size and the results are given in Table 1, below.

TABLE 1

| Particle size(μm) | Cumulative % |
|---|---|
| 0.985 | 0.07 |
| 1.89 | 12.41 |
| 2.50 | 32.52 |
| 3.31 | 53.23 |
| 4.38 | 66.82 |
| 5.27 | 77.28 |
| 6.35 | 85.32 |
| 11.11 | 95.69 |
| 21.32 | 98.96 |
| 78.56 | 100.00 |

### COMPARATIVE EXAMPLE 2

The dispersion prepared in Comparative Example 1 was treated at 7,000 psi with a high-pressure homogenizer, such as that manufactured by Microfluidics, identified as “Microfluidizer M110EHI”, in one pass. The particles thus obtained were analyzed for size and the results are given in Table 2, below. The resulting dispersion was measured to have a transmittance at 700 nm of 0.16%.

TABLE 2

| Particle Size(μm) | Cumulative % |
|---|---|
| 0.985 | 0.03 |
| 1.89 | 11.25 |
| 2.50 | 30.43 |
| 3.31 | 54.47 |
| 4.38 | 66.55 |
| 5.27 | 79.74 |
| 6.35 | 88.45 |
| 11.11 | 96.21 |
| 21.32 | 99.46 |
| 94.65 | 100.00 |

### EXAMPLES 1 TO 8

In 1 liter vessel, plant sterol (sitosterol 75%, campesterol 10%, and stigmasterol and sitostanol 15%), sucrose stearyl ester (HLB 11) and sorbitan lauryl ester (HLB 8.6) were mixed as shown in Table 3, below, and melted at 140° C. with stirring. After completion of the melting, the solution was stirred further for 1 min and added to water heated to about 80° C., followed by stirring at 6,800-7,000 rpm for about 10 min. In the case of Examples 2, 4, 6 and 8, the resulting solution was further treated at 7,000 psi with a high-pressure homogenizer (Microfluidizer M110EHI, Microfluidics) in one pass.

The solution of Example 1 was analyzed for particle size distribution and the results are given in Table 4, below. The analysis results of the particles of Examples 3, 5 and 7 were similar to those shown in Table 4. Particles of Example 2 were analyzed for size distribution and the results are given in Table 5, below. The analysis results of the particles of Examples 4, 6, and 8 were similar to those shown in Table 5. The plant sterol dispersions prepared in Examples 2, 4, 6 and 8 were measured to range, in transmittance at 700 nm, from 80.0 to 80.5%.

TABLE 3

| Exmp. | Plant | Sucrose stearyl | Sorbitan | | High pressure |
|---|---|---|---|---|---|
| No. | Sterol | Ester | lauryl Ester | H₂O | Homogenization |
| 1 | 5 g | 4.25 g | — | 500 g | Not done |
| 2 | 5 g | 4.25 g | — | 500 g | Done |
| 3 | 5 g | 3.036 g | 1.214 g | 500 g | Not done |
| 4 | 5 g | 3.036 g | 1.214 g | 500 g | Done |
| 5 | 25 g | 2.5 g | 1.75 g | 500 g | Not done |
| 6 | 25 g | 2.5 g | 1.75 g | 500 g | Done |
| 7 | 25 g | 2.126 g | 2.124 g | 500 g | Not done |
| 8 | 25 g | 2.126 g | 2.124 g | 500 g | Done |

TABLE 4

| Particle Size(μm) | Cumulative % |
|---|---|
| 0.096 | 20.35 |
| 0.127 | 52.19 |
| 0.153 | 68.49 |
| 0.184 | 75.29 |
| 0.222 | 85.33 |
| 0.294 | 91.52 |
| 0.985 | 99.21 |
| 5.27 | 100.0 |

TABLE 5

| Particle Size(μm) | Cumulative % |
|---|---|
| 0.096 | 13.67 |
| 0.127 | 49.40 |
| 0.153 | 69.39 |
| 0.184 | 77.61 |
| 0.222 | 89.07 |
| 0.294 | 95.22 |
| 0.985 | 99.89 |
| 2.08 | 100.0 |

In 1 liter vessel, plant sterol (sitosterol 75%, campesterol 10%, and stigmasterol and sitostanol 15%) and sucrose stearyl ester (HLB 11) and sorbitan lauryl ester (HLB 8.6) were mixed as shown in Table 6, below, and melted at 140° C. with stirring. After completion of the melting, the solution was further stirred for 1 min and added to a solution of 1 g of sucrose stearyl ester in water (80° C.), followed by stirring at 6,800-7,000 rpm for about 10 min. In the case of Examples 10, 12, 14 and 16, the resulting solution was treated at 7,000 psi with a high-pressure homogenizer (Microfluidizer M110EHI, Microfluidics) in one pass.

The solution of Example 9 was analyzed for particle size distribution and the results are given in Table 7, below. The analysis results of the particles of Examples 11, 13 and 15 were similar to those shown in Table 7. Particles of Example 10 were analyzed for size distribution and the results are given in Table 8, below. The analysis results of the particles of Examples 12, 14, and 16 were similar to those shown in Table 8. The plant sterol dispersions prepared in Examples 10, 12, 14 and 16 were measured to range, in transmittance at 700 nm, from 80.5 to 82.5%.

TABLE 6

| Exmp. | Plant | Sucrose stearyl | Sorbitan | | High-pressure |
|---|---|---|---|---|---|
| No.. | Sterol | ester | lauryl ester | H₂O | Homogenization |
| 9 | 5 g | 4.25 g | — | 500 g | Not done |
| 10 | 5 g | 4.25 g | — | 500 g | Done |
| 11 | 5 g | 3.036 g | 1.214 g | 500 g | Not done |
| 12 | 5 g | 3.036 g | 1.214 g | 500 g | Done |
| 13 | 25 g | 2.5 g | 1.75 g | 500 g | Not done |
| 14 | 25 g | 2.5 g | 1.75 g | 500 g | Done |
| 15 | 25 g | 2.126 g | 2.124 g | 500 g | Not done |
| 16 | 25 g | 2.126 g | 2.124 g | 500 g | Done |

TABLE 7

| Particle Size(μm) | Cumulative % |
|---|---|
| 0.096 | 19.21 |
| 0.127 | 52.30 |
| 0.153 | 68.72 |
| 0.184 | 76.41 |
| 0.222 | 85.95 |
| 0.294 | 92.05 |
| 0.985 | 99.35 |
| 4.80 | 100.0 |

TABLE 8

| Particle Size(μm) | Cumulative % |
|---|---|
| 0.096 | 14.50 |
| 0.127 | 48.24 |
| 0.153 | 70.68 |
| 0.184 | 77.92 |
| 0.222 | 90.61 |
| 0.294 | 96.74 |
| 0.985 | 99.85 |
| 1.89 | 100.0 |

### EXAMPLE 17

In 1 liter vessel, 5 g of plant sterol (sitosterol 75%, campesterol 10%, and stigmasterol and sitostanol 15%) and 4.25 g of polyglycerine monostearate (HLB 12) were melted at 140° C. with stirring. After completion of the melting, the melt was further stirred for 1 min and added to 490.75 g of water heated to about 80° C., followed by stirring at 6,800-7,000 rpm for about 10 min. The resulting solution was treated at 7,000 psi with a high-pressure homogenizer (Microfluidizer M110EHI, Microfluidics) in one pass.

Results from particle size analysis before the high-pressure homogenization were the same as in Table 4 within allowable experimental errors (2%). The same results as in Table 5 were found in the particle size analysis, within allowable experimental errors, after the high-pressure homogenization. The plant sterol dispersion after the high-pressure homogenization was measured to range, in transmittance at 700 nm, from 80.0 to 80.5%.

### EXAMPLE 18

In 1 liter vessel, 5 g of plant sterol (sitosterol 75%, campesterol 10%, and stigmasterol and sitostanol 15%) and 3.25 g of polyglycerine monostearate (HLB 12) were melted at 140° C. with stirring. After completion of the melting, the melt was further stirred for 1 min and added to 491.25 g of water heated to about 80° C., followed by stirring at 6,800-7,000 rpm for about 10 min. The resulting solution was treated at 7,000 psi with a high-pressure homogenizer (Microfluidizer M110EHI, Microfluidics) in one pass.

Results from particle size analysis before the high-pressure homogenization were the same as in Table 7 within allowable experimental errors (2%), while the same results as in Table 8 were found in the particle size analysis, within allowable experimental errors, after the high-pressure homogenization. The plant sterol dispersion after the high-pressure homogenization was measured to range, in transmittance at 700 nm, from 80.2 to 82.5%.

### EXAMPLE 19

In 1 liter vessel, 5 g of plant sterol (sitosterol 75%, campesterol 10%, and stigmasterol and sitostanol 15%) and 4.25 g of sucrose stearyl ester (HLB 11) were melted at 140° C. with stirring. After completion of the melting, the melt was further stirred for 1 min and added to 500 g of water heated to about 80° C., followed by stirring at 6,800-7,000 rpm for about 10 min. The resulting dispersion was spray-dried to give water-soluble plant sterol powder.

### EXAMPLE 20

In 1 liter vessel, 5 g of plant sterol (melting point 143° C.) and 4.25 g of sucrose stearyl ester (HLB 11) were melted at 140° C. with stirring. After completion of the melting, the melt was further stirred for 1 min and cooled to room temperature to give a solid which was then pulverized into powders. 9.25 g of powders was dispersed 90.75 g of water heated to about 90° C., with stirring at 6,800-7,000 rpm for about 10 min. The resulting dispersion was treated at 7,000 psi with a high-pressure homogenizer (Microfluidizer M110EHI, Microfluidics) in one pass.

### EXAMPLE 21

Preparation of Plant Sterol-Containing Yogurt

60% of heat-sterilized raw milk, 10% of each of the dispersions given in Table 9, below, 5% of nonfat dry milk, 7% of an oligosaccharide, 2% of a stabilizer, and 16% of water (on the weight basis) were homogeneously mixed at 65° C. The mixture thus obtained was passed at 210 kg/cm² through a homogenizer to form small, uniform particles. The mixture was then sterilized by heating at 95° C. for 10 min and cooled to 42° C. After being inoculated in an amount of 0.01% into the mixture, lactic acid starter was cultured at 42° C. When the pH of the culture was reduced to 4.6, the culture was cooled to 10° C. to stop the growth of the bacteria, and aged for 6 hours at the same temperature. The culture was mixed at a ratio of 75:25 with a previously heat-sterilized fruit syrup, immediately after which the mixture was packed in an oxygen-tight vessel and stored in a refrigerator. After storage at 4° C. for 30 days, the yogurt was observed for water separation, phase separation and taste change, and the observation results are given in Table 9, below.

TABLE 9

| | | Water | Phase | Taste |
|---|---|---|---|---|
| No. | Dispersion | Separation | Separation | Change |
| 1 | Prepared in Example 1 | N. O | N. O | N. O |
| 2 | Prepared in Example 2 | N. O | N. O | N. O |
| 3 | Prepared in Example 3 | N. O | N. O | N. O |
| 4 | Prepared in Example 4 | N. O | N. O | N. O |
| 5 | Prepared in Example 9 | N. O | N. O | N. O |
| 6 | Prepared in Example 10 | N. O | N. O | N. O |
| 7 | Prepared in Example 18 | N. O | N. O | N. O |
| 8 | Prepared in Example 19 | N. O | N. O | N. O |
| 9 | Prepared in Example 20 | N. O | N. O | N. O |

N. O: not observed.

### EXAMPLE 22

### Preparation of Plant Sterol-Containing Gruel

Along with 7.5 g of rice, 10.0 g of glutinous rice was washed with water and soaked in cold water for 4 hours. After being dewatered, the rice mixture was ground, and heated in 73.5 g of water in a double bath 3 g of starch was added to the rice gruel which was then further heated for 10 min to the point of a proper viscosity. 5.8 g of each of the dispersions shown in Table 10, below and 0.2 g of salt were mixed with the rice gruel which was packed at a predetermined amount in heat-resistant packs and sterilized at 130° C. for 30 min in a retort sterilizer. After storage at room temperature for 3 months then at 55° C. for 5 days, the rice gruel in packs was observed for water separation, phase separation and taste, and the results are given in Table 10, below.

TABLE 9

| | | Water | Phase | Taste |
|---|---|---|---|---|
| No. | Dispersion | Separation | Separation | Change |
| 1 | Prepared in Example 1 | N. O | N. O | N. O |
| 2 | Prepared in Example 2 | N. O. | N. O. | N. O. |
| 3 | Prepared in Example 3 | N. O. | N. O. | N. O. |
| 4 | Prepared in Example 4 | N. O. | N. O. | N. O. |
| 5 | Prepared in Example 9 | N. O. | N. O. | N. O. |
| 6 | Prepared in Example 10 | N. O. | N. O. | N. O. |
| 7 | Prepared in Example 18 | N. O. | N. O. | N. O. |
| 8 | Prepared in Example 19 | N. O. | N. O. | N. O. |
| 9 | Prepared in Example 20 | N. O. | N. O. | N. O. |

N. O.: not observed.

### INDUSTRIAL APPLICABILITY

As described hereinbefore, plant sterol particles dispersed in foods are such a size that they are greatly improved in bio-availability, leading to a decrease in serum cholesterol level even with little ingestion. Additionally, the plant sterol nano-sized particles are not bristly in the mouth, and have no influence on the characteristic taste and flavor of the foods. Further, the plant sterol nanoparticles can be applied to almost all food irrespective of beverage bases and pH. Also, the plant sterol particles do not show water separation and phase separation, guaranteeing the stability of the products for a long period of time.

1. A method for preparing a plant sterol-containing food, comprising the steps of:
thermally melting an admixture of plant sterol and at least one emulsifier at 60-200° C., said emulsifier being selected from the group consisting of sucrose fatty acid esters, sorbitan fatty acid esters and polyglycerine fatty acid esters;
combining the molten admixture with water or emulsifier-containing water;
stirring the combination at a high speed to give a dispersion of plant sterol of micelle form in water; and
applying the dispersion to a food base, the plant sterol being dispersed into particles with a size of hundreds of nanometers or less in the food base.

2. A method for preparing a plant sterol-containing food, comprising the steps of:
thermally melting an admixture of plant sterol and an emulsifier at 60-200° C., said emulsifier being selected from the group consisting of sucrose fatty acid esters, sorbitan fatty acid esters and polyglycerine fatty acid esters;
combining the molten admixture with water or emulsifier-containing water;
stirring the combination at a high speed, followed by homogenizing to give a dispersion of plant sterol of micelle form in water; and
applying the dispersion to a food base, the plant sterol being dispersed into particles with a size of hundreds of nanometers or less in the food base.

3. A method for preparing a plant sterol-containing food, comprising the steps of:
thermally melting an admixture of plant sterol and at least one emulsifier at 60-200° C., said emulsifier being selected from the group consisting of sucrose fatty acid esters, sorbitan fatty acid esters and polyglycerine fatty acid esters;
cooling the molten admixture for solidification, pulverizing the solidified admixture into powders, and combining the powders with water or emulsifier-containing water;
stirring the combination at a high speed to give a dispersion of plant sterol of micelle form in water; and
applying the dispersion to a food base, the plant sterol being dispersed into particles with a size of hundreds of nanometers or less in the food base.

4. A method for preparing a plant sterol-containing food, comprising the steps of:
thermally melting an admixture of plant sterol and at least one emulsifier at 60-200° C., said emulsifier being selected from the group consisting of sucrose fatty acid esters, sorbitan fatty acid esters and polyglycerine fatty acid esters;
cooling the molten admixture for solidification, pulverizing the solidified admixture into powders, and combining the powders with water or emulsifier-containing water;
stirring the combination at a high speed, followed by homogenizing to give a dispersion of plant sterol of micelle form in water; and
applying the dispersion to a food base, the plant sterol being dispersed into particles with a size of hundreds of nanometers or less in the food base.

5. The method as set forth in claim 1, wherein 95.0% or more of the dispersed plant sterol particles have a size of 300 nanometers or less.

6. The method as set forth in claim 5, wherein 99.0% or more of the dispersed plant sterol particles have a size of 300 nanometers or less.

7. The method as set forth in claim 1, wherein the emulsifier is a sucrose fatty acid ester.

8. The method as set forth in claim 7, wherein the sucrose fatty acid ester has a hydrophilic lipophilic balance value of 7 or higher.

9. The method as set forth in claim 8, wherein the sucrose fatty acid ester has a hydrophilic lipophilic balance value of 10 to 16.

10. The method as set forth in claim 1, wherein the emulsifier is a polyglycerine fatty acid ester.

11. The method as set forth in claim 10, wherein the polyglycerine fatty acid ester has a hydrophilic lipophilic balance value of 10-20.

12. The method as set forth in claim 11, wherein the polyglycerine fatty acid ester has a hydrophilic lipophilic balance value of 12-15.

13. A plant sterol-containing food prepared by the method of claim 1.

14. The food as set forth in claim 13, wherein the food is yogurt, gruel, soup, ice cream, mayonnaise, ketchup, salad oil, dressings, or margarine.

15. The method as set forth in claim 2, wherein 95.0% or more of the dispersed plant sterol particles have a size of 300 nanometers or less.

16. The method as set forth in claim 15, wherein 99.0% or more of the dispersed plant sterol particles have a size of 300 nanometers or less.

17. The method as set forth in claim 3, wherein 95.0% or more of the dispersed plant sterol particles have a size of 300 nanometers or less.

18. The method as set forth in claim 17, wherein 99.0% or more of the dispersed plant sterol particles have a size of 300 nanometers or less.

19. The method as set forth in claim 4, wherein 95.0% or more of the dispersed plant sterol particles have a size of 300 nanometers or less.

20. The method as set forth in claim 19, wherein 99.0% or more of the dispersed plant sterol particles have a size of 300 nanometers or less.

21. The method as set forth in claim 2, wherein the emulsifier is a sucrose fatty acid ester.

22. The method as set forth in claim 21, wherein the sucrose fatty acid ester has a hydrophilic lipophilic balance value of 7 or higher.

23. The method as set forth in claim 22, wherein the sucrose fatty acid ester has a hydrophilic lipophilic balance value of 10 to 16.

24. The method as set forth in claim 3, wherein the emulsifier is a sucrose fatty acid ester.

25. The method as set forth in claim 24, wherein the sucrose fatty acid ester has a hydrophilic lipophilic balance value of 7 or higher.

26. The method as set forth in claim 25, wherein the sucrose fatty acid ester has a hydrophilic lipophilic balance value of 10 to 16.

27. The method as set forth in claim 4, wherein the emulsifier is a sucrose fatty acid ester.

28. The method as set forth in claim 27, wherein the sucrose fatty acid ester has a hydrophilic lipophilic balance value of 7 or higher.

29. The method as set forth in claim 28, wherein the sucrose fatty acid ester has a hydrophilic lipophilic balance value of 10 to 16.

30. The method as set forth in claim 2, wherein the emulsifier is a polyglycerine fatty acid ester.

31. The method as set forth in claim 30, wherein the polyglycerine fatty acid ester has a hydrophilic lipophilic balance value of 10-20.

32. The method as set forth in claim 31, wherein the polyglycerine fatty acid ester has a hydrophilic lipophilic balance value of 12-15.

33. The method as set forth in claim 3, wherein the emulsifier is a polyglycerine fatty acid ester.

34. The method as set forth in claim 33, wherein the polyglycerine fatty acid ester has a hydrophilic lipophilic balance value of 10-20.

35. The method as set forth in claim 34, wherein the polyglycerine fatty acid ester has a hydrophilic lipophilic balance value of 12-15.

36. The method as set forth in claim 4, wherein the emulsifier is a polyglycerine fatty acid ester.

37. The method as set forth in claim 36, wherein the polyglycerine fatty acid ester has a hydrophilic lipophilic balance value of 10-20.

38. The method as set forth in claim 37, wherein the polyglycerine fatty acid ester has a hydrophilic lipophilic balance value of 12-15.

39. A plant sterol-containing food prepared by the method of claim 2.

40. The food as set forth in claim 39, wherein the food is yogurt, gruel, soup, ice cream, mayonnaise, ketchup, salad oil, dressing, or margarine.

41. A plant sterol-containing food prepared by the method of claim 3.

42. The food as set forth in claim 41, wherein the food is yogurt, gruel, soup, ice cream, mayonnaise, ketchup, salad oil, dressing, or margarine.

43. A plant sterol-containing food prepared by the method of claim 4.

44. The food as set forth in claim 43, wherein the food is yogurt, gruel, soup, ice cream, mayonnaise, ketchup, salad oil, dressing, or margarine.
